(19) United States
(12) Patent Application Publication
(10) Pub. No.: US 2010/0278762 A1
(43) Pub. Date: Nov. 4, 2010

(54) LONG-ACTING, WATERPROOF OR WATER-RESISTANT, TOPICAL SUN PROTECTION AGENT WITH ACTIVITY UP TO WEEKS

(76) Inventors: Hilton M. Kaplan, Los Angeles, CA (US); Julie R. Kailua, Koohoo Place Kailua, HI (US)

Correspondence Address: Louis C. Paul;Louis C. Paul & Associates, PLLC, 150 East 58th Street, 34th Floor, New York, NY, 10155, US

(21) Appl. No.: 12/741,029

(22) PCT Filed: Oct. 30, 2008
(86) PCT No.: PCT/US2008/081876

§ 371 Date: May 1, 2010

Related U.S. Application Data
(60) Provisional application No. 60/984,649, filed on Nov. 1, 2007.

(51) Int. Cl. A61K 33/30; A61Q 17/04

(52) U.S. Cl. 424/59

(57) ABSTRACT

Disclosed herein are compositions for use as a sunscreen or sunblock which, in some embodiments, may provide long-acting, waterproof or water-resistant, broad spectrum topical sun protection with activity up to weeks without reapplication. In some embodiments, the compositions may include a base and one or more active ingredients. In some embodiments, the base may include an adhesive component, which upon application to a user's skin, adheres the base to the user's skin. In some embodiments, the adhesive component is adapted to hold the active ingredients on the user's skin for at least about 8 hours, such as at least about 24 hours, such as at least about 3 days, or even at least about 2 weeks. Furthermore, in some embodiments, the composition may be waterproof or water-resistant, In some embodiments, the one or more active ingredients may include a photodamage protective ingredient, and/or a photodamage reparative ingredient.

### BACKGROUND OF THE INVENTION

1. Field of the Invention

This application relates to long-lasting sunblock or sunscreen compositions that, in some embodiments, may provide sun-protection for a period of days to weeks without reapplication. In some embodiments the sunblock or sunscreen may be waterproof or water-resistant.

2. Description of the Related Art

The dangers of free radical production by ultraviolet (UV) light exposure have become an enormous public health issue, worsened by the deleterious effects of greenhouse gases on the ozone layer and global warming. In 2000, the NIH listed solar radiation as a known human carcinogen for the first time.¹

UV radiation is divided into three categories according to wavelength: UVA rays range from ˜320-400 nm (nanometers), UVB rays from ˜290-320 nm, and UVC rays from ˜100-290 nm. Both UVA and UVB are found in sunlight, while UVC is completely filtered by the ozone layer. UVA is ˜100 times more prevalent than UVB; and because UVA has the longest wavelengths, it penetrates the skin more deeply than UVB. UVA is subdivided into two wavelength ranges which are known as UVA-I (˜340-400 nm) and UVA-II (˜320-340 nm). While all UV light directly interacts with the skin's DNA to induce mutations, the primary method of destruction is the formation of reactive oxygen species (ROS), destructive molecules known as a “free radicals”, which cause cumulative damage to healthy cells, including cells of the immune system.

Non-melanoma skin cancer is by far the most common cancer seen in man. Melanoma, the most deadly form of skin cancer, has recently been identified as the cancer with the sharpest increase in prevalence among all cancers. UV light is a primary carcinogen in non-melanoma skin cancers, and a co-carcinogen in melanomas. Most of the more than 1 million eases of non-melanoma skin cancer diagnosed in the United States each year are sun-related.² With ozone depletion progressing and recreational time in the sun increasing, the rates of skin cancer are expected to increase in the future, despite already being at epidemic levels in certain countries such as Australia. Accordingly, the development of improved sunscreens and sunblocks is critical to protect against the deleterious health effects of UV light.

### SUMMARY OF THE INVENTION

Disclosed herein are compositions for use as a sunscreen or sunblock. In some embodiments, the compositions may include a base, and one or more active ingredients. In some embodiments, the base may include an adhesive component. In some embodiments, upon application to a user's skin, the adhesive component adheres the base to the user's skin. In some embodiments, the adhesive component is adapted to hold the active ingredients on the user's skin for at least about 8 hours. In some embodiments, the adhesive component is adapted to hold the active ingredients on the user's skin for at least about 24 hours. In some embodiments, the adhesive component is adapted to hold the active ingredients on the user's skin for at least about 3 days. In some embodiments, the adhesive component is adapted to hold the active ingredients on the user's skin for at least about 2 weeks.

In some embodiments, the adhesive component includes a first polymer. In certain such embodiments, the first polymer may be made up of acrylate monomers. In certain such embodiments, the first polymer may be made up of acrylamide monomers. In certain such embodiments, the first polymer may be made up of vinyl acetate monomers.

In some embodiments, the composition may include a film-forming non-adhesive component. In certain embodiments, the adhesive component and the film-forming non-adhesive component undergo phase separation after the composition is applied to the user's skin. In certain embodiments, the weight ratio of the adhesive component to the film-forming non-adhesive component may be between about 8:1 and about 1:8.

The film-forming non-adhesive component may include a second polymer. In certain such embodiments, the second polymer may include acrylate monomers, or vinyl acetate monomers, or one or more types of monomers comprising a siloxane moiety, a fluorine moiety, or any combination of the preceding monomers.

The compositions may also include a volatile solvent.

The compositions also include one or more active ingredients. In some embodiments, the active ingredients include a photodamage protective ingredient. In some embodiments, the active ingredients include a photodamage reparative ingredient. In certain such embodiments an active ingredient may be both protective and reparative. In certain embodiments, the active ingredients may include a photodamage protective ingredient, and a separate photodamage reparative ingredient. In certain embodiments the photodamage protective and/or reparative ingredient includes a metal oxide, which may or may not be micronized/microfine. In certain embodiments, the metal oxide may be an oxide of titanium, or an oxide of zinc, or both. In certain embodiments the photodamage protective and/or reparative ingredient includes melanin. In certain embodiments, the photodamage protective and/or reparative ingredient may include an antioxidant such as enzogenol. In certain embodiments, the active ingredients may include a sunless tanning agent such as dihydroxyacetone. In certain embodiments, the active ingredients may include a sebum absorber such as fumed silica.

In certain embodiments the compositions may also include aesthetic modifying ingredients such as one or more pigments or one or more rheological agents.

Also disclosed herein are methods of applying various sunblock or sunscreen compositions to a person's skin to protect the skin against the effects of sun-exposure. The various sunblock or sunscreen compositions applied to the skin may comprise one or more embodiments of the inventive compositions disclosed herein.

For example, in one embodiment, a method of protecting a person's skin against the effects of sun-exposure comprises applying a fluid onto the person's skin, the fluid comprising a sunblock or sunscreen composition. In some embodiments, applying the fluid may comprise spraying the fluid onto the person's skin. In some embodiments, applying the fluid onto the person's skin may comprise topical painting of the fluid onto the person's skin, topical wiping of the fluid onto the person's skin, topical rubbing of the fluid onto the person's skin, and/or topical massaging of the fluid onto the person's skin. In some embodiments, such a method may include the step of drying the fluid after the fluid has been applied to the user's skin.

### DETAILED DESCRIPTION OF THE PREFERRED EMBODIMENTS

For the last several decades, health protection against the damaging effects of solar radiation was advocated by using sunscreens which protect against sunburns, a UVB induced-phenomenon. The longevity of that protection (provided that adequate amounts were applied and the product wasn't washed off) was indicated by the SPF rating. The sun protection factor (SPF) is the factor of time that it takes to detect erythema (redness) on the skin with sunscreen on relative to without it. The SPF rating is a specific UVB monitor, with precise guidelines put forth by the FDA. Unfortunately, while UVB is the most energetic (see (*) below) of the UV rays that come through the atmosphere, and its deleterious effects on the skin are readily apparent, the UVB rays make up only a small portion of the available UV light (<5%). Furthermore, UVB is readily blocked by most materials, including glass to varying degrees, and penetrates skin only superficially.

(*) The energy associated with the electromagnetic spectrum is proportional to its frequency; such that the energy carried by a photon of radiation of given wavelength is described by the following relationship:

$$Δ E = h \frac{c}{λ} = h υ$$

Where E=energy, h=Planck's constant, c=velocity of light, λ=wavelength, and v=frequency.

In contrast, UVA (˜320-400 nm) makes up 95% of the solar radiation coming through the atmosphere, and as UVA has a longer wavelength than UVB it penetrates more deeply into the dermis, and readily may bypass windows, cloud cover, rain, and many fabrics. UVA protection of sunscreen can be measured in vivo from immediate pigment darkening (IPD) and/or persistent pigment darkening (PPD: an immediate but lasting response of 2-24 hours) due to photo-oxidation of melanin (caused by UVA action spectrum of 320-400 nm at dose of 8-25 J/cm²); or in vitro by determination of the critical wavelength (an international rating system for UVA protection: the point at which a sunscreen allows 10% of rays to penetrate; a critical wavelength >370 nm is considered excellent UVA protection by the FDA). Yet until recently UVA had no specific FDA monitoring guidelines, FDA regulations still remain unfinalized, and UVA protection in sunblocks has yet to be routinely rated for commercial consumers. The damaging health effects of UVA are becoming ever more evident, from carcinogenicity, to skin immunosuppression, to premature aging of skin. It is this last area, the so called “photoaging” effects of UVA which has brought UVA to the attention of consumers, with sunscreen manufacturers anxious to state that their product contains protection against UVA. Unfortunately however, without a uniform rating system, degrees of UVA protection largely remain unknown. The carcinogenic effects of UVA are also becoming better understood, and there is discussion as to whether the protection against only UVB radiation in the past has led people to incur more UVA exposure than would have been the case, had the sunburn been allowed to occur (as with adequate UVB protection people do not burn noticeably, and so stay in the sun longer due to this false sense of security—so acquiring more UVA damage than if their exposures were more limited by the erythema and discomfort associated with UVB burning).

Therefore, to protect against the deleterious health effects of ultraviolet light, it is useful to partially or substantially block both UVA and UVB wavelengths. Further, regarding sunblock characterization, a reliable and consistent measure of UVA protection should ideally compliment the SPF method used for measuring UVB protection.

Sunscreens have traditionally been split into two broad categories: the chemical (organic) sunscreens and the physical (inorganic) sunblocks. Note that, while the inventors recognize the distinction between “sunblocks” and “sunscreens,” one of ordinary skill in the art would recognize that, in many cases, or for many purposes, sunblocks and sunscreens can be used interchangeably. Accordingly, in this disclosure, use of either “sunblock” or “sunscreen” is not meant to imply that the other is excluded. Chemical sunscreens partially “block” the penetration of UV radiation through the epidermis by acting as filters: absorbing high energy UV and then releasing this energy as lower-energy rays (heat). Chemical sunscreens have, for a long time, provided some protection against UVB, but have only more recently been developed to provide a portion of UVA protection as well. Chemical sunscreens have activity within certain wavelengths, which tend to be more narrowly within the UVB or UVA spectrum. Under regulatory standards, sunscreen ingredients must comply with stringent levels of stability. During stability testing, the ingredients are subjected to intense UV radiation, which is partially absorbed and scattered by them before it can reach the skin. In some embodiments, energy may be filtered by a sunscreen agent through reflection, diffraction, and/or absorption (e.g. conversion to a safer wavelength of energy such as heat). Some chemical ingredients form by-products that can decrease the active ingredient's integrity, create toxicity (free radicals; allergens) in the skin, and/or completely break down and fail the user. The fact that many chemical sunscreens themselves can be altered by the very light and/or heat that they are designed to protect against, and so decompose with time, is not widely recognized by consumers; who tend to keep sunscreen bottles in the heat at the beach or in a car, and may have a single bottle for several years (expiration dates are not always routinely labeled on sunscreens). The important Riverside report³ recently documented that certain chemical sunscreens inadvertently increase skin damage due to the creation of free radicals at the surface of the skin from the ingredients themselves. It is also well known that many chemical sunscreens can induce direct or photo-provoked allergenicity, either whole or as breakdown byproducts.

Physical sunblocks usually contain opaque materials, such as, for example, materials derived from ground minerals, which substantially sit on the skin's surface. Light is either partially absorbed into the sunblock material or partially reflected away from the body back into the atmosphere similar to a mirror or tin foil. Ideally, physical sunblocks are effective at protecting against both UVA and UVB radiation within a broad range. Two common physical blockers are titanium dioxide (TiO₂) and zinc oxide (ZnO). These agents are excellent sunscreens as they are relatively chemically inert, relatively safe, and substantially protect against a relatively substantial range of the UV spectrum. They are usually not associated with allergic reactions. Therefore physical sunscreens work relatively well for people with sensitive skin and children. At one time, these sunblocks could be readily identifiable as a thick, greasy, opaque white film, but new technology has created these sunblocks with relatively tiny particles (micronized or ultrafine grades). By the reduction of the particle size, these agents somewhat lose their capacity to reflect and scatter light in the visible wavelength range, and so they are relatively transparent and thus more cosmetically acceptable. These smaller particles acquire absorptive capacity in the UV wavelength range, and so the particles often attenuate UV radiation predominantly by absorption somewhat similar to an organic sunscreen. Because physical sunblocks are often minerals, these products don't tend to decompose, and they often are not susceptible to substantial degradation by the elements.

The risk of sundamage is also very importantly influenced by lack of reapplication of sunscreen (i.e. poor compliance). This can be due to physical restrictions such as sweating; abrasive removal of the sunscreen by clothing, waves, etc; dislike for inelegant, uncomfortable formulations such as greasy or thick creams; or simply not having access to, or remembering to reapply sunscreens as frequently as they should be reapplied. The American Academy of Dermatology supports the statement that sunscreens should be reapplied every 2 hours, a practice which is poorly or not followed by the majority of consumers. Recently, in an attempt to address this limitation, newer, relatively photostable, chemical UVA and UVB sunscreens have been developed to, in some cases, provide photoprotection for up to ˜4 hours (detailed below). It remains unclear how these chemical sunscreens hold up to prolonged storage or the physical elements such as heat, light and water. It is also not yet known if the chronic application of these chemicals on the skin over prolonged periods will produce chemical degradation and byproducts (with the potential to cause free radical damage or allergenicity).

Certain sunblock or sunscreen compositions disclosed herein potentially outlast this current state of the art of ˜4 hours maximum photoprotection, in excess of ˜2 fold better (˜8 hrs), in excess of ˜6 fold better (˜24 hrs), and, in some embodiments, in excess of ˜18-30 fold or better (3-5+ days).

In certain embodiments, the sunblock or sunscreen compositions disclosed herein provide photodamage protection after application to a user's skin for at least about 8 hours, or at least about 12 hours, or at least about 16 hours, or at least about 20 hours, or at least about 24 hours, or at least about 36 hours, or at least about 2 days, or at least about 3 days, or at least about 4 days, or at least about 5 days, or at least about 6 days, or at least about 7 days, or at least about 2 weeks, or at least about 3 weeks, also including durations between these recited durations.

In some embodiments, the length of time the disclosed sunblock or sunscreen compositions provide photodamage protection varies according to many factors. These factors may include, but are not limited to, the skin-type of the particular person using the composition, the type of activities engaged in by the person using the composition, the location on the person's body to which the composition is applied, and so on. For example, a person engaged in athletic activities may perspire heavily and their perspiration may alter the length of time an embodiment remains on the person's skin. In some instances, abrasion of an area of the user's skin to which the user has applied the composition may affect an embodiment's longevity. For example, such abrasion could occur due to characteristics of the user's clothing, or because of contact with dirt or sand or other materials. Furthermore, certain embodiments may remain effective for longer periods on certain areas of a user's body than on other areas, due to the fact, for example, that people's skin tends to produce oils and/or perspire more in some areas than in other areas. Accordingly, in some embodiments, the addition of one or more sebum absorbers to a sunblock/sunscreen composition may increase the timeframe during which a sunblock/sunscreen composition remains effective on the skin.

In certain embodiments, the compositions disclosed herein address the need for long lasting sun protection by utilizing the relatively well defined, and relatively stable, and relatively broad spectrum UV light reflection of the physical sunblocks. Some formulations may also include antioxidant and/or quenching ingredients to counter rogue free radical damage, as well as optional vitamin ingredients to potentially repair previous solar radiation damage. All these advantages may be combined in a relatively long lasting breathable film, which, in some embodiments, may last in the order of 8 hours, to several days, to several weeks. The ability to limit incidental or accidental solar radiation exposure by offering continuous protection potentially on the order of days to weeks, will help to block and dramatically lessen this major carcinogen implicated in the causation of skin cancers as well as premature aging.

Agents which protect against both UVA as well as UVB include: broad spectrum chemical UVA+UVB filters with increased photostability such as Tinosorb™ (chemical class hydroxyphenyltriazine (HPT)) in M (water soluble) and S (oil soluble) forms; as well as physical (inorganic and insoluble) sunscreens such as micronized zinc oxide (ZnO) and microfine titanium dioxide (TiO₂).

As used herein, a micronized/microfine/ultrafine substance refers to a substance wherein the majority of particles comprising the substance have diameters on the order of microns/micrometers (μm) or less—e.g. diameters of less than 1000 μm. Thus, for example: a substance micronized to 1000 μm or less refers to a substance wherein the majority of particles comprising the substance have diameters of 1000 μm or less; a substance micronized to 100 μm or less refers to a substance wherein the majority of particles comprising the substance have diameters of 100 μm or less; a substance micronized to 10 microns or less refers to a substance wherein the majority of particles comprising the substance have diameters of 10 μm or less.

A substance can be reduced to nanoparticles through milling or other methods known to those skilled in the art. Thus, a substance in the form of nanoparticles refers to a substance wherein the majority of particles comprising the substance have diameters on the order of nanometers (nm) or less—e.g. diameters of less than 1000 nm. Thus, for example: a substance in the form of 1000 nm nanoparticles refers to a substance wherein the majority of particles comprising the substance have diameters of 1000 nm or less; a substance in the form of 100 nm nanoparticles refers to a substance wherein the majority of particles comprising the substance have diameters of 100 nm or less; a substance in the form of 10 nm nanoparticles refers to a substance wherein the majority of particles comprising the substance have diameters of 10 nm or less. Note, that as these terms are used herein, a substance that has been reduced to nanoparticles has also been micronized; however, a substance that has been micronized has not necessarily been reduced to nanoparticles.

ZnO and TiO₂ are both relatively inert (though TiO₂ more so than ZnO), and can be formulated so that neither substantially reacts with other ingredients such as vitamins or antioxidants. These metal oxides absorb and scatter across a relatively broad range of wavelengths in comparison to some of the other sunblocks or sunscreens which are currently available. Microfine or nano-sized ZnO is primarily a UV light absorber and captures a broader spectrum of rays than many other ingredients (generally, as the particle size used increases, it produces progressively less absorption and more diffraction and reflection of UV light). It is the only sunscreen or sunblock agent that we know of that is capable of blocking the longer UVA-II rays. The use of ZnO as a sunscreen is internationally widely allowed. In the USA, it can be used at levels of 2% to 25%, alone or in combination with other sunscreen ingredients. It is an extremely well tolerated ingredient, with an estimated five million pounds used on human skin each year in the USA alone. ZnO is relatively stable when exposed to UV light, and does not substantially break down or produce by-products when exposed to heat and light (and so is unlikely to induce allergenicity or the substantial formation of free radicals). It is also classified as non-comedogenic (i.e. it will not induce acne).⁴ Zn is also an important antioxidant for the skin, and so it may act not only as a sunscreen ingredient but also as a photoprotectant.^{4,5} Furthermore, for nearly a century Zn ointments have been a common natural skin care treatment for conditions as broad-ranging as psoriasis, eczema, dermatitis, seborrhea, seborrheic dermatitis, dandruff, poison oak, poison ivy, athlete's foot, acne, diaper rash, cradle cap, etc.

An example of a newer chemical sunscreen lasting in the 4 hour range is Helioplex™ (Neutrogena, Johnson & Johnson), comprised of:
homosalate, octisalate, octocrylene (photostable UVB filters);
oxybenzone (photostable UVB and short UVA filter);
avobenzone (photostabilized long UVA filter);
diethylhexyl 2,6 naphthalate (DEHN) (stabilizer—energy transfer molecule).

Other possible sun protection ingredients include (INCI name in parentheses):⁶
Photostable: UVB: Parsol SLX (polysilicone-15 (dimethicodiethylbenzal malonate));
   UVA: Neo Heliopan AP (bis-disulizole disodium);
      Uvinul A Plus (diethylamino hydroxybenzoyl hexyl benzoate).
   UVA+UVB: Mexoryl SX, Ecamsule, Silatriazole (terephthalylidene dicamphor sulfonic acid): a photostable short UVA filter.
Photolabile: UVB: Padimate O (ethylhexyl dimethyl para-amino-benzoic-acid (PABA));
   Octinoxate (ethylhexyl methoxycinnamate).

UVA Parsol 1789, Avobenzone (butyl methoxydibenzoylmethane).

### Potential Effects of Prolonged Sun Protection

### Sun Exposure and Vitamin D

The question of whether prolonged use of sunscreens has any negative influence on vitamin D levels and health has been studied extensively, with the following conclusions:^{7,8,9,10,11,12,13,14}
1. The action spectrum for skin production of vitamin D and the acute and chronic carcinogenic effects of UVB are the same.
2. With long term sunscreen use, levels of parathyroid hormone, calcium, and bone mass density remain unaltered.^{10,11,12,13,14}
3. For individuals with fair skin, vitamin D photosynthesis is maximal after only very modest sun exposure.
4. A less than adequate serum vitamin D level in some individuals (i.e. in winter, elderly, darkly pigmented individuals), should be addressed with diet and supplementation.
5. Adequate vitamin D levels are maintained with: incidental sun exposure, dietary intake, vitamin supplementation.
6. It is inappropriate to recommend intentional sun exposure for achieving adequate vitamin D levels due to the greater risk of promoting skin cancer.

### Sun Exposure and Melanoma Risk

The incidence of invasive malignant melanoma (MM) in the US in 2006 was ˜62,190 (vs. 250,000 squamous cell carcinomas (SCC) and 1,000,000 basal cell carcinomas (BCC)). All recent data with national data sets show that the overall MM incidence is increasing. Currently MM rates are rising faster than for any other cancer in the US. Prevention and early detection are still the best “treatment and cure”.

Melanoma risk factors:
Dysplastic nevi
Personal history of MM
Family history of MM
Fair skin, light eyes, blond/red hair
Number of sunburns (especially prior to age 21)*
Outdoor summer jobs as teenager*
Freckles*
Many nevi* *Of the above list, the last 4 risks could be reduced by enhanced sun protection.

The inventors have considered various methods for applying ingredients to the skin more intensely and/or deeply than by simple topical application alone, and have considered specifically the potential of sunscreen ingredients in this regard; together, in some cases, with additional ingredients that are protective and/or reparative (one of the inventors' goals of these combinations is defined here as to “repair-while-protecting”). Many potential methods have been explored, and the resultant idea of a semi-permanent or long-lasting sunscreen/sunblock was eventually born. In order to design a long-acting, possibly waterproof or water-resistant, topical sunscreen with activity in the order of 8 hours, to days, to weeks, ingredients and formulas based on the following characteristics have been researched:

### 1) Base:

In order to act as a carrier of the active ingredients, in some embodiments the base preferably has one or more of the following characteristics:
i. Relatively inert, yet would still allow the active ingredients and other ingredients to work.
ii. Easy to apply.
iii. Waterproof or water-resistant, yet relatively easy to remove if desired.
iv. Relatively durable, staying intact for 8 hours, to days, to weeks, yet comfortable to wear and relatively easy to remove if desired.
v. Safe in terms of toxicity, allergenicity and comedogenicity when remaining on the skin for up to 8 hours, to days, to weeks.

To find the appropriate base, experiments were conducted with a variety of glue ingredients from Elmers™ to Dermabond™ medical grade tissue glue/cyanoacrylates. Ultimately glues consisting of a variety of potential acrylate mixtures were explored.

### 2) Active Ingredients:

The active ingredients may include (but are not limited to) one or more of the following (and where there are both photodamage protective ingredients and photodamage reparative ingredients, the formulation can be said to have properties of “repair-while-protecting”):
i. Photodamage protective ingredients, such as both UVA and UVB sunblocks, that are effective in the base, that preferably remain substantially activated over a period of 8 hours, to days, to weeks, that preferably do not substantially interfere with any other ingredients, and that preferably are not substantially interfered with by other ingredients.
ii. Photodamage reparative ingredients, such as antioxidants and/or quenchers, that are effective in the base, that preferably remain substantially effective for at least hours, to days, to weeks, that preferably do not substantially interfere with any other ingredients, and that preferably are not substantially interfered with by other ingredients.
iii. Additional topical active ingredients may be included as desired, for example: self-tan/sunless tanning agents which may make the formulation more desirable to the user and hence, in some instances, more effective through added compliance, and/or may be employed to offer a visual indicator of the longevity of the product on the skin; sebum absorbers which, in some embodiments, may enhance the duration that the product will remain on the skin for; etc.
iv. Preservatives, if desirable, safe, and beneficial.

Metal oxides such as ZnO and TiO2 were investigated and experimented with as these physical sunblocks not only offer relatively broad spectrum protection against both UVA & UVB light, but also remain substantially effective, and substantially biologically and chemically inert over relatively long periods of time (unlike some chemical sunscreen absorbers which can be broken down by light and/or heat in a matter of minutes to hours and have the potential to create free radicals on/in the skin (as explained above) and to induce cutaneous allergic reactions).

A variety of potential antioxidant ingredients substantially meeting the above criteria were investigated. Several are listed as ingredients in the embodiments below.

### 3) Aesthetic Modifying Ingredients:

In order to add user-friendliness to the formulation, one or more additional components can be included, such as, but not limited to:
i. Pigments: a variety of pigments, added to achieve flesh-colored tones, the appearance of transparency to some extent, or bright colors as are often favored by youth and sports enthusiasts. The pigments not only may enhance the aesthetic quality of the product, but may also be employed to offer a visual indicator of the longevity of the product on the skin.
ii. Rheological Agents: a variety of rheological agents, added to create the desired embodiment such as a spray, a cream, a gel, or a lotion, etc. In order to create a spray for example, ingredients may be added to the formulation to allow aerosolizability, without substantially compromising efficacy or durability; with packaging appropriate to this mode of application.

Some of the formulations disclosed herein provide one or more benefits relative to existing sunblocks to date, such as:
1. Reducing the dosing requirement from ˜2-4 hours to every ˜8 hrs-7 days, or even to weeks.
2. Improving compliance of sunscreen use, thereby lessening the incidence of sunburn/unintended sun exposure, and eventually positively impacting skin cancer rates and premature aging.
3. Improving compliance in occupations that involve the use of masks/goggles, which are compromised by using traditional cream sunscreen formulations.
4. Improved effectiveness in hot climates or activities where sweating occurs and conventional sunscreens run and come off.
5. Reducing the occurrence of sunscreen dripping into and irritating the eyes.
6. Reducing sunscreen allergies and induced acne by being non-allergenic and non-comedogenic.
7. Potentially reducing the risk of pathogens (bacteria, viruses, fungi) from gaining entry into the skin by enhancing the barrier effects of skin and by inherent antimicrobial properties (e.g. Zn).
8. Reducing incidental sun exposure from windows, cloud cover, etc. in situations where sunscreens conventionally may not have been employed.
9. Enhancing water resistance relative to conventional sunblocks. Possibly waterproof.
10. Greater resistance to abrasion from soap and water, such as in bathing/handwashing and repeated wiping with towels.
11. Reducing the impact of sunscreens being washed off in the oceans and leading to the death of corals and/or other life forms.^{15,16}

Accordingly, in preferred embodiments, the formulations and methods of use provide one or more of these 11 benefits.

The compositions disclosed herein, may comprise one or more of the following constituents/components:

### 1) Base:

Based upon several of the preferred attributes detailed above, in one embodiment, the base is a relatively low viscosity mixture of acrylates, to be sprayed on, or otherwise applied, as a film. Certain formulations of acrylates are known to be safe when used as wound closure glues and dressings and, in some cases, have even been shown to promote would healing. Numerous “liquid bandages” which are available on the market today are based on such. Furthermore, certain acrylates, such as certain cyanoacrylates, are hypoallergenic and non-comedogenic (cyanoacrylate strips are in fact used as a formal test to assess comedogenicity in a method called “cyanoacrylate follicular biopsy”). Mixtures of acrylates may be formulated at a low viscosity so that the formulation will be easy to apply, such as it may be applied to form a uniform film; and is easy to remove such as by dissolution through reapplication, or the use of a variety of known non-toxic solvents which dissolve the solute base (e.g. mineral oil), or the use of an exfoliant. Some formulations of acrylates exhibit sufficient durability to last on the skin for days to weeks.

Formulations suitable for use as a base include, but are not limited to, compositions similar to that taught in U.S. Pat. No. 6,958,154 B2 (“Spray On Bandage and Drug Delivery System”) which is incorporated by reference in its entirety herein. However the formulations disclosed herein are not specifically formulated to serve as drug delivery systems, but rather to hold active ingredients at the surface of the skin so that they may, in some embodiments, form a sunscreen barrier there, and/or hold other active ingredients to act on such surface, such as antioxidants, for example.

The FDA's determination of water resistance is as “water resistant” if a topical application is able to withstand 2×20 min water immersions (separated by 20 minutes of air drying); and as “very water resistant” if it is able to withstand 4×20 min such water immersions. In some embodiments, the formulation would be able to withstand repeated water immersions and washings over periods on the order of 8 hours, to days, to weeks.

Several possible base formulations are described in U.S. Pat. Nos. 6,958,154; 4,751,087; 4,737,577; Re 24,906; and 7,097,853—each incorporated by reference in their entirety herein in which the base is a fluid composition, e.g. an aerosol spray, which is applied onto the surface as a fluid and then dries to form a non-adhesive outer layer over an underlying adhesive layer that adheres the base onto the surface, e.g. the skin. In certain embodiments, the formulations include an adhesive component and a film-forming, non-adhesive component, which are substantially immiscible with each other so that the components undergo substantial phase separation as the fluid composition dries rapidly. Note, that the term “fluid” as used throughout herein includes, but is not limited to, creams, oils, lotions, aerosols (gases), etc.

In certain embodiments, the adhesive component comprises a pressure sensitive adhesive comprising an acrylate (or methacrylate) polymer, where the polymer is a polymer of monomers comprising: (1) about 40%-50%, 50%-60%, 60%-70%, 70%-80%, 80%-90%, or about 90%-99% by weight (and combinations thereof) of an alkyl (meth)acrylate (of 1 to 10 carbon atoms); and (2) about 1%-10%, 10%-20%, 20%-30%, 30%-40%, 40%-50%, or about 50%-60% by weight (and combinations thereof) of a free radically copolymerizable monomer (with a functional group selected from carboxylic acid, carboxylic acid ester, hydroxyl, anhydride, epoxy, thiol, isocyanate, sulfonamide, urea, carbamate, carboxamide, amine, ammonium, oxy, oxo, nitro, nitrogen, sulfur, phosphate, phosphonate, cyano, and combinations thereof).

In certain embodiments, the adhesive component comprises a pressure sensitive adhesive comprising an acrylate (or methacrylate) polymer, where the polymer is a polymer of monomers comprising: (1) between about 40%-99% by weight of an alkyl (meth)acrylate (of 1 to 10 carbon atoms), including about 50%, 60%, 70%, 80%, or about 90% by weight (and also including weight percentages between these recited percentages, as well as ranges of weight percentages bordered on each end by the recited weight percentages); and (2) between about 1% and 60% by weight of a free radically copolymerizable monomer (with a functional group selected from carboxylic acid, carboxylic acid ester, hydroxyl, anhydride, epoxy, thiol, isocyanate, sulfonamide, urea, carbamate, carboxamide, amine, ammonium, oxy, oxo, nitro, nitrogen, sulfur, phosphate, phosphonate, cyano, and combinations thereof), including about 10%, 20%, 30%, 40%, and about 50% by weight (and also including weight percentages between these recited percentages, as well as ranges of weight percentages bordered on each end by the recited weight percentages).

In certain embodiments, the adhesive component comprises a pressure sensitive adhesive comprising an acrylate (or methacrylate) polymer, where the polymer is a polymer of monomers comprising: (1) between about 40%-99%, or about 50-90%, or about 60-80% by weight of an alkyl (meth)acrylate (of 1 to 10 carbon atoms); and (2) between about 1%-60%, or about 10%-50%, or about 20%-40% by weight of a free radically copolymerizable monomer (with a functional group selected from carboxylic acid, carboxylic acid ester, hydroxyl, anhydride, epoxy, thiol, isocyanate, sulfonamide, urea, carbamate, carboxamide, amine, ammonium, oxy, oxo, nitro, nitrogen, sulfur, phosphate, phosphonate, cyano, and combinations thereof).

The adhesive component may also comprise a polymer derived from monomers comprising: (1) about 40%-50%, 50%-60%, 60%-70%, 70%-80%, 80%-90%, or about 90%-99.9% by weight (and combinations thereof) of isooctyl (meth)acrylate; and (2) about 0.1%-10%, 10%-20%, 20%-30%, 30%-40%, 40%-50%, or about 50%-60% by weight (and combinations thereof) of (meth)acrylamide; and (3) about 0%-0.1%, 0.1%-10%, 10%-20%, or about 20%-30% by weight (and combinations thereof) of vinyl acetate.

The adhesive component may also comprise a polymer derived from monomers comprising: (1) between about 40% to about 99% by weight of isooctyl (meth)acrylate, including about 50%, 60%, 70%, 80%, and about 90% by weight (also including weight percentages between these recited percentages, as well as ranges of weight percentages bordered on the high end and the low end by the recited weight percentages); and (2) between about 0.1% to about 60% by weight of (meth)acrylamide, including about 10%, 20%, 30%, 40%, and about 50% by weight (also including weight percentages between these recited weight percentages, as well as ranges of weight percentages bordered on the high side and the low side by the recited weight percentages); and (3) between about 0.1% and about 30% by weight of vinyl acetate, including 10%, and about 20% by weight (also including weight percentages between these recited weight percentages, as well as ranges of weight percentages bordered on the high side and the low side by the recited weight percentages).

The adhesive component may also comprise a polymer derived from monomers comprising: (1) between about 40%-99%, or about 50%-90%, or about 60%-80% by weight of isooctyl (meth)acrylate; and (2) between about 0.1%-60%, or about 10%-50%, or about 20%-40% by weight of (meth)acrylamide; and (3) between about 0.1%-30%, or about 10%-20% by weight of vinyl acetate.

The adhesive component may also comprise an acrylate pressure sensitive adhesive comprising a polymer formed by copolymerizing: (1) about 60%-70%, or about 70%-80%, or about 65%-75% by weight (and combinations thereof) of isooctyl acrylate; and (2) about 1%-5%, or about 5%-10%, or about 2.5%-7.5% by weight (and combinations thereof) of acrylamide; and (3) about 5%-10%, or about 10%-20%, or about 20%-30%, or about 5%-30%, or about 10%-25%, or about 15%-20% by weight (and combinations thereof) of vinyl acetate.

Other suitable adhesive materials in the class of (meth)acrylate polymers are those embodiments described in U.S. Pat. Nos. 4,751,087; 4,737,577; Re 24,906; and 7,097,853—each incorporated by reference in their entirety herein. These adhesives may be prepared by free-radical polymerization methods known in the art, including but not limited to bulk, solution, emulsion and suspension polymerization methods. For example, according to the solution polymerization method, polymers suitable for use in the formulation are prepared by dissolving the desired monomers in an appropriate solvent, adding a chain-transfer agent, a free-radical polymerization initiator, and other additives known in the art, sealing the solution in an inert atmosphere such as nitrogen or argon, and then agitating the mixture at a temperature sufficient to activate the initiator.

The non-adhesive, film-forming component is substantially “incompatible” or substantially “immiscible” with the adhesive component, in that a solution comprising the adhesive and non-adhesive components undergoes substantial phase separation during drying, meaning that during drying the two components substantially separate from each other to form a bi-layer film. The layers may or may not have a complete separation—there may be an interface portion between the layers where there is some mixing. In some embodiments, the non-adhesive component may comprise at least one low surface energy, surface seeking moiety, wherein said film-forming, non-adhesive component is substantially incompatible with the adhesive component, and wherein the film-forming, non-adhesive component is present in an effective amount such that upon application it undergoes substantial phase separation from the adhesive component such that an outer surface of the in-situ formed covering element is substantially non-adhesive when the covering element is touch dry. A polymer comprising at least one siloxane moiety and/or at least one fluorine containing moiety, and/or acrylate monomers, and/or vinyl acetate monomers, and/or acrylamide monomers are one suitable non-adhesive component. Examples of polymers suitable for some embodiments include: a siloxane containing polymer, a polyvinylacetate, a polymethyl(meth)acrylate, a fluorinated polymer, a fluorosilicone polymer, a styrene-butadiene rubber, a polyurethane, a vinyl copolymer, a polyolefin, a polyamide, a polyimide, a polyamidemeide, a polyester, and combinations of these.

The weight ratio of the adhesive component to the non-adhesive component is between about 1:25 to about 25:1 including about 1:8, 1:5, 2:5, 3:5, 4:5, 1:1, 5:4, 5:3, 5:2, 5:1, and about 8:1, and also including ratios between these recited ratios, as well as ranges of ratios bordered on each end by the recited ratios. Thus, possible ranges of weight ratios of adhesive to non-adhesive components include, but are not limited to: 1:25-25:1, 1:8-8:1, 1:5-5:1, 2:5-5:2, 3:5-5:3, and 4:5-5:4. However, non-symmetric ranges are also possible.

Note that the term acrylate is used herein in the broadest sense understood to one of ordinary skill in the art, and includes for example (without limitation), what are referred to as methacrylates, methyl acrylates, methyl methacrylates, methyl(meth)acrylates, and cyanoacrylates. Also, note that the term polymer is used herein in the broadest sense understood to one of ordinary skill in the art, and includes for example (without limitation), what are often referred to as copolymers and terpolymers, and generally includes any composition consisting of polymerized monomers.

Certain sunscreen or sunblock compositions disclosed herein comprise a base and one or more active ingredients. In some embodiments, the base comprises an adhesive component, wherein upon application to a user's skin, the adhesive component adheres the base to the user's skin. In certain such embodiments, the base further comprises a film-forming non-adhesive component, wherein the adhesive component and the film-forming non-adhesive component undergo phase separation after the composition is applied to the user's skin. In certain such embodiments, after application, the adhesive component is adherent against/to the user's skin and the film-forming non-adhesive component is on top of the adhesive component.

The adhesive component may comprise a first polymer. In some embodiments, the first polymer may comprise acrylate monomers, acrylamide monomers, vinyl acetate monomers, or any combination of these monomers. In some embodiments, the first polymer may comprise alkyl (meth)acrylate monomers comprising 1 to 10 carbon atoms, and/or monomers comprising a functional group selected from carboxylic acid, carboxylic acid ester, hydroxyl, anhydride, epoxy, thiol, isocyanate, sulfonamide, urea, carbamate, carboxamide, amine, ammonium, oxy, oxo, nitro, nitrogen, sulfur, phosphate, phosphonate, and cyano, so long as the monomers are polymerizable with each other.

In some embodiments, upon application of the composition to a user's skin, the adhesive component adheres the composition to the user's skin and is adapted to hold the active ingredients on the user's skin for at least about 8 hours, or at least about 12 hours, or at least about 16 hours, or at least about 20 hours, or at least about 24 hours, or at least about 36 hours, or at least about 2 days, or at least about 3 days, or at least about 4 days, or at least about 5 days, or at least about 6 days, or at least about 7 days, or at least about 2 weeks, or at least about 3 weeks.

It is to be understood, however, that for each duration recited in the previous paragraph, it is not necessarily the case that, in every embodiment, substantially all of the applied active ingredients remain on the user's skin for the full recited duration. Depending on the embodiment, the percentage of active ingredients that remain adhered to the user's skin for the full recited duration may be about 95%, 90%, 80%, 70%, 60%, 50%, 40%, 30%, 20%, and about 10% (including percentages between these recited percentages, and also including ranges of percentages bordered on the high side and the low side by the recited percentages). Thus, for example, a composition adapted to hold the active ingredients on the user's skin for at least about 2 days, may hold between about 50% and 70% of the applied active ingredients on the user's skin for at least about 2 days, depending on the embodiment. However, it is also to be understood that, generally speaking, to hold the active ingredients on the user's skin for a recited duration means to hold some (more than an insubstantial amount) of the active ingredients on the user's skin for the recited duration. Furthermore, it is also to be understood, that for each duration recited in the previous paragraph, it is not necessarily the case that, in every embodiment, the SPF of the applied active ingredients remains the same for the full recited duration. Depending on the embodiment, the SPF may vary from the initial SPF by some percentage such as about 95%, 90%, 80%, 70%, 60%, 50%, 40%, 30%, 20%, and about 10% of the initial SPF (including percentages between these recited percentages, and also including ranges of percentages bordered on the high side and the low side by the recited percentages). Thus, for example, a composition adapted to hold the active ingredients on the user's skin for at least about 2 days, may retain an SPF of between about 50% and 70% of the initial SPF for at least about 2 days, depending on the embodiment.

The film-forming non-adhesive component may comprise a second polymer. In some embodiments, the second polymer may comprise acrylate monomers, vinyl acetate monomers, and/or one or more types of monomers comprising a siloxane moiety, a fluorine moiety, or both.

In some embodiments, the base includes one or more volatile solvents such that the fluid composition has a viscosity allowing the fluid composition to be coated or sprayed onto said surface. Suitable volatile solvents include, but are not limited to, ethanol, acetone, isopropanol, or a combination thereof. In some embodiments, fluid compositions generally may comprise between about 5% to about 40% by weight of solvent based upon the total weight of the composition, including about 10%, 20%, and about 30% by weight, also including weight percentages between these recited percentages, as well as ranges of percentages bordered on each end by the recited percentages.

In some embodiments, volatile solvents are therapeutically safe and skin tolerant. Such solvents may also, although not necessarily, be nonflammable, yet may have a sufficiently high vapor pressure under ambient conditions such that a coating of the fluid composition is touch dry within 10 minutes, including within 3 minutes, including within 30 seconds, after being coated onto a surface. Such solvents may solvate at least one, or all, of the components of the fluid composition so that the composition can be coated or sprayed onto the desired surface as a homogeneous slurry, dispersion, or solution, as the case may be. In some embodiments, isopropanol is one example of a suitable solvent. A co-solvent is also possible in some embodiments. One example is a co-solvent comprising: (1) between about 1% and 100% parts by weight ethanol, including about 10%, 20%, 30%, 40%, 50%, 60%, 70%, 80%, and about 90%, also including percentages between these recited percentages, as well as ranges of percentages bordered on each end by the recited percentages; and (2) between about 1% and 100% parts by weight hexamethyldisiloxane, including about 10%, 20%, 30%, 40%, 50%, 60%, 70%, 80%, and about 90%, also including percentages between these recited percentages, as well as ranges of percentages bordered on each end by the recited percentages.

The base can either be made from scratch as detailed in this section and under some of the sample embodiments below; or any of a number of commercially available formulations may be used as a base, with or without alteration, some examples of which are listed here: Band-Aid™ Single-Step Liquid Bandage (Johnson & Johnson Consumer Products, Skillman, N.J.; includes 2-octylcyanoacrylate, siloxane plasticizer); New-Skin™ Liquid Bandage (Medtech, Jackson, Wyo.; includes ethanol/SD alcohol 40, isopropanol, butyl acetate, ethyl acetate, amyl acetate, nitrocellulose); Nexcare™ Spray Liquid Bandage (3M, St Paul, Minn.; includes hexamethyldisiloxane, acrylate terpolymer, polyphenylmethylsiloxane copolymer); Skin Shield™ Liquid Bandage (Del Pharmaceuticals, Uniondale, N.Y.; includes ethanol/SD alcohol 40, acetone, ethyl acetate, nitrocellulose).

### 2) Active Ingredients:

One or more of the following active ingredients may be included in a formulation, in order to provide sun protection and/or “repair-while-protecting”:

i. Photodamage Protective Ingredients:

Both the metal oxides ZnO and TiO₂ are substantially inert physical sunblocks and offer relatively broad spectrum protection against both UVA and UVB solar radiation. Metal oxides as sunblock reflectants/absorbers remain inert and active over long periods. ZnO has additional cutaneous contact benefits such as aiding in wound healing and improving scarring, as well as functioning in the cutaneous immune system. Furthermore, micronized metal oxides are commercially available to be nearly invisible on the skin if desired. As used herein, a micronized substance refers to a substance wherein the majority of particles comprising the substance have diameters on the order of microns or less—i.e. diameters of less than 1000 μm.

The FDA defines SPF as:

$$SPF = \frac{MED\_protected \_skin}{MED\_unprotected \_skin}$$

Where MED=minimal erythematous dose, as determined using a solar simulator at 290-400 nm, applied between beltline and scapula, with each test site ≧50 cm² (10 cm×5 cm or 4″×2″), and using 2 mg/cm² of formulation on the protected site (which is equivalent to 1 oz of formulation being required to cover the entire body surface).

For a complete block applied for 3-7 days, the potential SPF value exceeds 288 and is conceivably in excess of 672+ (based upon an MED of 15 minutes for Fitzpatrick skin type I remaining protected for 24 hours per day for more than 3-7 days).

In one embodiment, the sunscreen active agent is micronized/nano-sized zinc oxide (ZnO) in particle sizes in the range of, by example only, ˜0.01-200 μm. As the particle size used increases, it generally produces progressively less absorption and generally more diffraction and reflection of UV light. ZnO filters a broad spectrum of rays compared to other ingredients, and is capable of blocking the longer UVA-II rays. Its use as a sunscreen is allowed widely internationally. In the USA it can be used at levels of about 2% to about 25% by weight, alone or in combination with other sunscreen ingredients. It is an extremely well tolerated ingredient, with an estimate of five million pounds used on human skin each year in the USA alone. ZnO is stable when exposed to UV light, and does not substantially break down or produce by-products when exposed to heat and light (and so is unlikely to induce allergenicity or the substantial formation of free radicals). It is also classified as non-comedogenic (will not induce acne).¹⁷ Zn is also an important antioxidant for the skin, and so it may act not only as a sunscreen ingredient but also as a photoprotectant.^{4,18} Furthermore, for nearly a century Zn ointments have been a common natural skin care treatment for conditions as broad-ranging as psoriasis, eczema, dermatitis, seborrhea, seborrheic dermatitis, dandruff, poison oak, poison ivy, athlete's foot, acne, diaper rash, cradle cap, etc.

Some of the sunscreen/sunblock embodiments disclosed herein may comprise a dispersion comprising ZnO in the form of suspended particles in about 2% to about 25% by weight (including about 5%, 10%, 15%, and about 20%, also including weight percentages between these recited percentages, as well as ranges of percentages bordered on each end by the recited percentages), and other active agents in the form of suspended particles; the total suspended particles being present in an amount of from about 1% to about 70% by weight of suspended particles (including about 5%, 10%, 20%, 30%, 40%, 50%, and about 60%, also including percentages between these recited percentages, as well as ranges of percentages bordered on each end by the recited percentages), based upon 100% weight of total dispersion.

In other embodiments, the sunscreen active agent may be titanium dioxide (TiO₂). TiO₂ is the world's most widely utilized white pigment. Its high refractive index, ease of dispensability and photochemical stability are unique. For this reason it is used to impart opacity and whiteness to an enormous variety of products including paints, plastics, inks, paper, cosmetics, foodstuffs and pharmaceuticals. At a particle size of about 300 μm the maximal visible light is scattered and the appearance is bright white; to avoid this, particles can be milled to a smaller size including a size of less than 1 μm (1000 nm), i.e. to “nanoparticles”. Smaller particles may be useful for sunscreens as they are colorless at that size and yet can still reflect both UVA and UVB.

Some of the sunscreen/sunblock embodiments disclosed herein may comprise a dispersion comprising the sunscreen TiO₂ in the form of suspended particles or nanoparticles in about 1% to about 50% by weight (including about 5%, 10%, 20%, 30%, and about 40%, also including percentages between these recited percentages, as well as ranges of percentages bordered on each end by the recited percentages), and other active agents in the form of suspended particles; the total suspended particles being present in an amount of from about 1% to about 70% by weight of suspended particles (including about 5%, 10%, 20%, 30%, 40%, 50%, and about 60%, also including percentages between these recited percentages, as well as ranges of percentages bordered on each end by the recited percentages), based upon 100% weight of total dispersion.

In some of the sunscreen/sunblock embodiments disclosed herein, a photodamage protective ingredient may comprise a combination of ZnO and TiO₂, where a dispersion comprises ZnO in the form of suspended particles in about 0.5% to about 25% by weight (including about 1%, 2%, 5%, 10%, 15%, and about 20%, also including percentages between these recited percentages, as well as ranges of percentages bordered on each end by the recited percentages), and the sunscreen TiO₂ in the form of suspended particles or nanoparticles in about 0.5% to about 50% by weight (including about 1%, 2%, 5%, 10%, 20%, 30%, and about 40%, also including percentages between these recited percentages, as well as ranges of percentages bordered on each end by the recited percentages), and other active agents in the form of suspended particles; the total suspended particles being present in an amount of from about 1% to about 70% by weight of suspended particles (including about 2%, 5%, 10%, 20%, 30%, 40%, 50%, and about 60%, also including percentages between these recited percentages, as well as ranges of percentages bordered on each end by the recited percentages), based upon 100% weight of total dispersion.

In some embodiments, the active ingredients comprise, alone or in any variety of combinations, ingredients offering increased UVA protection, including but not limited to:
ZnO and TiO2 (in the aforementioned percentages).
Broad-spectrum UV filters such as Tinosorb S™ (bis-ethylhexyloxyphenol methoxyphenyl triazine) and/or Tinosorb M™ (methylene bis-benzotriazolyl tetramethylbutylphenol), and/or avobenzone (butyl methoxydibenzoylmethane), each in about 0.1%-20% by weight (including about 0.5%, 1%, 2%, 5%, 10%, and about 15%, also including weight percentages between these recited percentages, as well as ranges of percentages bordered on the high side and the low side by the recited percentages). Thus, in some embodiments, one possible range is 0.1-5.0% by weight. If these ingredients are included, stabilization of these ingredients may be useful to improve the safety and effectiveness of these ingredients over a period of several days.
A water-soluble benzotriazole such as Cibafast H™ (sodium benzotriazolyl butylphenol sulfonate (and) buteth-3 (and) tributyl citrate), in about 0.1%-20% by weight (including about 0.5%, 1%, 2%, 5%, 10%, and about 15%, also including weight percentages between these recited percentages, as well as ranges of percentages bordered on the high side and the low side by the recited percentages). Thus, in some embodiments, one possible range is 0.1%-5.0% by weight.
A water-soluble benzotriazole such as Tinogard HS™ (sodium benzotriazolyl butylphenol sulfonate), in about 1%-20% by weight (including about 0.5%, 1%, 2%, 5%, 10%, and about 15%, also including weight percentages between these recited percentages, as well as ranges of percentages bordered on the high side and the low side by the recited percentages). Thus, in some embodiments, one possible range is 0.1%-5.0% by weight.
An oil-soluble benzotriazole such as Tinogard TL™ (benzotriazolyl dodecyl p-cresol), in about 1%-20% by weight (including about 0.5%, 1%, 2%, 5%, 10%, and about 15%, also including weight percentages between these recited percentages, as well as ranges of percentages bordered on the high side and the low side by the recited percentages). Thus, in some embodiments, one possible range is 0.1%-5.0% by weight.
Melanin, which is a biological polymer with intrinsic ability to reduce free radical/ROS generation by UV radiation,¹⁹ in about 0.1%-10% by weight (including about 0.5%, 1%, 2%, 3%, 4%, 5%, 6%, 7%, 8%, and about 9%, also including percentages between these recited percentages, as well as ranges of percentages bordered on each end by the recited percentages). Thus, in some embodiments, one possible range is 0.1%-5.0% by weight.

ii. Photodamage Reparative Ingredients:

In order to repair oxidative stresses and mop up free radicals, a variety of vitamins, cofactors, minerals, antioxidants, quenchers or the like may be included.

Some examples of suitable topical vitamins include, but are not limited to: vitamin A as esters (such as retinyl palmitate, retinyl acetate), as alcohol (such as retinol), as acid (such as retinoic acid), as its natural plant form beta-carotene (retinaldehyde dimer); vitamin C as acid (ascorbic acid), as a salt (such as sodium or magnesium ascorbyl phosphate), as a fat (such as ascorbyl tetraisopalmitate), or as an ester-C; vitamin E as a fat (such as alpha tocopherol), or other; and the like.

An example of suitable topical cofactors includes, but is not limited to: coenzyme Q10 (CoQ10), and the like.

Examples of suitable topical minerals include, but are not limited to: zinc, magnesium, silicone and the like.

Some examples of suitable topical antioxidants and/or quenchers include, but are not limited to, alpha-lipoic acid, lipochromalin-6, idebenone, coffee berry, green tea polyphenols, Polypodium leucotomos (a fern plant extract), Tinogard TS™ (octadecyl di-t-butyl-4-hydroxyhydrocinnamate), Tinogard NOA™ (tetrabutyl ethylidinebisphenol), Tinogard Q™ (tris(tetramethylhydroxypiperidinol)citrate, an excited state quencher (ESQ) of molecules, or their excited-state intermediaries, such as UV blockers, e.g. avobenzone or the like),²⁰ manuka oil (from Leptospermum scoparium (manuka) honey), enzogenol (from Pinus radiata pine bark), and the like. Both of the latter 2 originate in New Zealand and have potent antioxidant and wound healing capabilities.

Optimally, the ingredients in this class compliment all the requirements of, and other ingredients in, the formulation; and also remain optimally active on the cutaneous/adherent under-surface of the film.

Accordingly, an embodiment may contain vitamins, cofactors, minerals, or antioxidants and/or quenchers in concentrations—either solely, or in combination with each other—of about 0.1% to about 10% by weight (including about 0.5%, 1%, 2%, 3%, 4%, 5%, 6%, 7%, 8%, and about 9%, also including percentages between these recited percentages, as well as ranges of percentages bordered on each end by the recited percentages).

Embodiments may contain the powerful and topically effective antioxidant enzogenol. Enzogenol is a proanthocyanidin extract from the bark of Pinus radiata, with the full phenolic compound spectrum. Enzogenol has been shown to protect the skin from both internal and external oxidation, improve blood circulation (nutrient delivery and toxin removal), and preserve connective tissue structure.

In some cases, enzogenol may reduce the internal peroxide load in human skin cells by more than 50%. Internal oxidative stress generated by normal cellular metabolism is greatly reduced & skin cells are protected from external oxidative stresses too. In some cases, enzogenol-protected cells demonstrate a 31% increase in growth compared to unprotected cells.

In some cases, enzogenol also protects skin cells from UV radiation, with, in some cases, enzogenol-protected cells demonstrating a 50% increase in growth in the face of 30 minutes of UV irradiation compared to unprotected cells.

Enzogenol can penetrate the skin (in some cases, 10% of the catechin component permeates in <6 hours; in some cases, 0.1% of the proanthocyanidins (OPCs) penetrates in <10 hours). At concentrations of 0.1%=1 mg/ml enzogenol/0.8 mg/ml OPCs, the concentration that could penetrate may be ˜1 μg/ml enzogenol. Skin cell experiments show that, in some cases, even 0.5 μg/ml enzogenol gives a very effective level for cell protection, so topical concentrations of 0.1% and higher may be effective based on the penetration rate of enzogenol OPCs (rat skin model).

Enzogenol can be included in an embodiment in an amount from about 0.1% to about 15% by weight (including about 1%, 2%, 5%, and about 10%, also including percentages between these recited percentages, as well as ranges of percentages bordered on each end by the recited percentages). One feasible range is between about 0.1%-5% by weight (for a desired application dose of 100-1000 mg per day of enzogenol, in a 150 ml total volume spray solution, which offers at least 6 large applications where each has a maximum enzogenol dose of about 1000 mg, the total amount of enzogenol required is 6 g per 150 ml total volume=4% by weight).

Some embodiments may comprise manuka oil in an amount of from about 0.1% to about 50% by weight (including about 0.5%, 1%, 2%, 5%, 10%, 20%, 30%, and about 40%, also including percentages between these recited percentages, as well as ranges of percentages bordered on each end by the recited percentages), but more preferable from about 0.1% to about 10% by weight, as taught in U.S. Pat. No. 6,956,144 (which is incorporated by reference in its entirety herein) and multiple others. Other embodiments comprising both enzogenol and manuka oil are also contemplated.

Another antioxidant that may be employed is Polypodium leucotomos^{21,22,23} (a fern plant extract from central America), which has been shown in both oral (Fernblock®) and topical forms to be photoprotective against UVB and PUVA-induced phototoxicity (PUVA=Psoralen+UVA treatment for eczema); as well as to be antioxidant and anti-inflammatory; with a dose dependent SPF of 25-90 (topical).

Accordingly, an embodiment may contain this plant extract in a concentration of about 0.1% to about 10% by weight (including about 0.5%, 1%, 2%, 3%, 4%, 5%, 6%, 7%, 8%, and about 9%, also including percentages between these recited percentages, as well as ranges of percentages bordered on each end by the recited percentages).

iii. Additional Topical Active Ingredients:

A self-tan/sunless tanning agent may be included in an embodiment to make it more desirable to the user and hence more effective through added compliance. dihydroxyacetone (DHA) is approved by the FDA as a cosmetic. It binds to basic proteins in keratin forming yellow-brown pigments (melanoidins) which have absorption in the UVA+visible light regions of the electromagnetic spectrum. In some embodiments, these stay bound to the skin for 3-10 days which is the length of time our formulae remain on the skin in certain embodiments, and so may also act as a marker of adequate protection. DHA does not substantially penetrate beyond the stratum corneum and in 2%-5% concentrations produces a sunless tan. Accordingly, an embodiment may contain DHA in a concentration of about 0.1% to about 6% by weight (including about 0.5%, 1%, 2%, 3%, 4%, and about 5%, also including percentages between these recited percentages, as well as ranges of percentages bordered on each end by the recited percentages). At 15% topical solution DHA has been reported to provide a photoprotective factor of 10 for UVA, however its use as a sunscreen has not been proven safe.

Sebum absorbers may be included, and in some embodiments, may reduce oiliness in certain skin types and/or body regions and so potentially may enhance the duration that the product will remain on the skin for. Suitable sebum absorbers may include, but are not limited to: calcium, magnesium or zirconium silicate: fumed silica; starches; clays; and vegetable powders. Some embodiments may comprise sebum absorbers in an amount of from about 0.1% to about 10% by weight (including about 0.5%, 1%, 2%, 3%, 4%, 5%, 6%, 7%, 8%, and about 9%, also including percentages between these recited percentages, as well as ranges of percentages bordered on each end by the recited percentages).

iv. Preservatives:

Certain embodiments may include preservatives. Suitable preservatives include, but are not limited to, chlorophenesin, sorbic acid, disodium ethylenedinitrilotetraacetate, phenoxyethanol, methylparaben, ethylparaben, propylparaben, phytic acid, imidazolidinyl urea, sodium dehydroacetate, benzyl alcohol, methylchloroisothiazolinone, methylisothiazolinone, and any combination of any of the foregoing.

Accordingly, an embodiment may contain from about 0.1% to about 20% by weight of preservatives—either as single preservatives, or in combination with each other (including about 0.2%, 0.3%, 0.4%, 0.5%, 0.7%, 1%, 1.5%, 2%, 2.5%, 5%, 10%, and about 15%, also including percentages between these recited percentages, as well as ranges of percentages bordered on each end by the recited percentages), based upon 100% weight of total sunscreen formulation.

### 3) Aesthetic Modifying Ingredients:

In order to add user-friendliness to the formulation, certain embodiments may include aesthetic modifying agents such as (but not limited to):
i. Pigments: a variety of pigments, added to achieve flesh-colored tones, the appearance of transparency, or bright colors as are often favored by youth and sports enthusiasts. The pigments not only enhance the aesthetic quality of the product, but may also be employed to offer a visual indicator of the longevity of the product on the skin. A variety of pigments can be added to achieve a range of flesh-colored tones, the appearance of transparency (using micronized metal oxides for example as described above), and/or bright colors.
ii. Rheological Agents: a variety of rheological agents, may be added to create an embodiment such as a spray, a cream, a gel, or a lotion, etc. In order to create a spray for example, ingredients may be added to the product to allow aerosolizability, without substantially compromising efficacy or durability; with packaging suitable to this mode of application. Appropriate viscosity solutions can facilitate a spray-on application which will dry quickly. In some embodiments, suitable rheological modifying agents from about 0.1% to about 60% by weight of the rheological modifying agent (including about 0.5%, 1%, 2%, 5%, 10%, 20%, 30%, 40%, and about 50%, also including percentages between these recited percentages, as well as ranges of percentages bordered on each end by the recited percentages), include, but are not limited to, a phosphorylated starch derivative (e.g. hydroxypropyl distarch phosphate), sodium hyaluronate, acrylates/C10-C30 alkyl acrylate crosspolymer, xanthum gum, cholesterol, hydroxypropyl distarch phosphate, carbomer, guar hydroxy propyltrimonium chloride, hydroxypropyl guar and sodium hydroxypropyl starch phosphate, etc. as taught by U.S. Pat. No. 7,175,834.

A method for creating a final formulation may include mixing of the ingredients together and storage in an airtight bottle with a spray applicator. In some embodiments, the solid ingredients will be suspended in the solution, which may require shaking or other agitation before application.

In some embodiments, making the fluid composition involves combining the components and mixing until a generally homogeneous solution, slurry, suspension, dispersion, gel, or the like as the case may be, is obtained. For example, to make some embodiments that can be sprayed onto the skin, the base is initially prepared by dissolving a silicone-urea block copolymer in the solvent isopropanol. The other ingredients of the composition such as the (meth)acrylates, active agents such as titanium dioxide or the like, and any of the optional aesthetic modifying agents are then added sequentially to the base and dissolved in the isopropyl alcohol and/or mixed into a suspension such as by paddle blade agitation to complete the formulation. This final formulation is then mixed with propeller agitation (or other suitable method) until homogenous. If a propellant is to be included, it may be added using techniques known in the art, e.g., cold filling or pressure filling techniques, after the other ingredients are placed into a suitable spray device.

Commercially available spraying systems may be used, such that a suitable coating thickness of between about 250 and 1000 μm is obtained (including about 350, 450, 550, 650, 750, 850, and about 950 μm, also including thicknesses between these recited thicknesses, as well as ranges of thicknesses bordered on each end by the recited thicknesses). In some embodiments, a thickness between about 250 and about 750 μm is obtained.

In some embodiments, the base film and the ingredients suspended within it can be removed from the host by simply pulling the covering element off the host surface without leaving a residue behind; or in some of the embodiments incorporating a (meth)acrylate adhesive, the covering element can be removed by swabbing or washing with a solvent such as isopropyl alcohol or mineral oil as the case may be to solvate a sufficient amount of the tacky phase to release the element from the skin.

In some embodiments, the spray bottle may be divided into 2 or more compartments to allow the long-acting sunscreen formulation to be available together with its solvent remover.

Disclosed herein are methods of applying various sunblock or sunscreen compositions to a person's skin to protect the skin against the effects of sun-exposure. The various sunblock or sunscreen compositions applied to the skin may comprise one or more embodiments of the inventive compositions disclosed herein.

For example, in some embodiments, methods of protecting a person's skin against the effects of sun-exposure comprise applying a fluid onto the person's skin, the fluid comprising a sunblock or sunscreen composition. In some embodiments, applying the fluid may comprise spraying the fluid onto the person's skin. In some embodiments, applying the fluid onto the person's skin may comprise topical painting of the fluid onto the person's skin, topical wiping of the fluid onto the person's skin, topical rubbing of the fluid onto the person's skin, and/or topical massaging of the fluid onto the person's skin, and/or other methods of topical application as are known in the art.

In some embodiments, such a method may include the step of drying the fluid after the fluid has been applied to the user's skin. In some embodiments, the drying is accomplished by simply allowing the fluid to dry naturally. In other embodiments, the drying may be accomplished through the use of a device to increase the flow of air or any gas over the applied fluid, thereby increasing the rate at which the fluid dries. In certain embodiments such a device is a mechanical fan. However, the device could simply be an implement used by a person to fan the applied fluid. In still other embodiments, the drying may be accomplished through the use of a device to heat the applied fluid, thereby increasing the rate at which the fluid dries. In yet other embodiments, a combination of heating and increasing air flow may be used to accelerate drying. In yet other embodiments, a combination of heating and moisture in the form of steam may be used to accelerate drying. In principle, any feasible method of accelerating the drying of the fluid may ease the use of certain embodiments of the sunscreen and/or sunblock compositions disclosed herein.

Initial testing of a total of 12 embodiments of the formulation was performed in order to direct and refine a formulation for full testing. The final chosen test embodiment (referred to as such below), representing a possible embodiment only, was more fully evaluated using:

1) In vitro and in vivo evaluation of UVA and UVB protection, using proposed FDA testing methodologies at an independent US-based sunscreen testing facility (Suncare Research Laboratories, LLC, Winston-Salem, N.C.).

2) A single-application week-long pilot clinical trial in adults and children: proof of concept testing on 7 subjects (2 children and 5 adults, skin types I-III) living near the equator was conducted to provide early data on the ability of a single application of the sunscreen to withstand high heat, humidity, and activity in differing skin-types and ages over 7 days.

3) Histological evaluation using standard hematoxylin and eosin (H&E) and polarization: histology was obtained from a single volunteer on day of sunscreen application and again after 5 days to evaluate localization in skin and demonstrate mechanism of action.

4) Toxicological review: ingredients were submitted for formal independent toxicological review (Regulatory and Technical Associates, Lebanon, N.J.), and institutional review board (IRB) approval (Allendale Investigational Research Board, Old Lyme, Conn.).

Results for the test embodiment are summarized as follows:

1) Performance data demonstrated that this test embodiment had broad spectrum UV protection with solid photostability (Static SPF 29.1, VWR (very water resistant) SPF 27.6, UVA Protection Factor 18.9, FDA (UVA1/UVA) Ratio 0.88=FDA Rating High, Boots (UVA/UVB) Ratio 0.81=Boots Star Rating 4*, and Critical Wavelength 383 nm).

2) The test embodiment was well tolerated by both adults and children, found to have the greatest longevity on skin in the non-oily parts of the body, and held up well to multiple daily showers and athletic activities including swimming. The test embodiment was found clinically to still be protective against sunburn at 4 days after single application. Volunteers rated the formulation comfortable and aesthetically acceptable. Despite its durability, the test embodiment was easily removed using mineral oil if desired.

3) Histology detailed the mechanism of action, demonstrating that the test embodiment was retained adherent in the outer layers of the stratum corneum, with normal shedding over time. The test embodiment was shown to remain substantially adherent to and within the surface layers of the dead stratum corneum both upon application and still at 5 days follow-up. This was clear both with and without polarization, but especially so with polarization. This demonstrates the mechanism of action as, in some embodiments at least partially, adherence to the stratum corneum, or elements within it; and its mechanism of release as, in some embodiments at least partially, separation of the stratum corneum with its attached composition, rather than solely by the composition itself coming loose (the latter mechanism appears likely, in some embodiments, to play a lesser role).

4) Toxicological and IRB review of the test embodiment confirmed the safety and non-toxic nature of the ingredients and applications.

In summary, these early data with respect to the test embodiment, demonstrate that sunscreen and/or sunblock compositions disclosed herein are able to block both UVA and UVB broadly for several days without the need for reapplication. The formulation used was aesthetically acceptable, hardy even in extreme heat and humidity, and determined to be safe and non-toxic. Importantly, no allergenicity nor comedogenicity was noted, nor was of concern on toxicological review. Formal patch testing and histological/rabbit ear testing are currently planned to further confirm non-allergenicity and non-comedogenicity respectively.

### Sample Embodiments

Some sample embodiments are provided here, but any combination of these or any of the ingredients listed in this disclosure can be used to form suitable embodiments by those skilled in the art.

### Sample Embodiment 1

### 1) Base

a. Adhesive component: polymer of about 60% to about 80% isooctyl acrylate, about 1% to about 10% acrylamide, and about 5% to about 30% vinyl acetate.
b. Non-adhesive component: polymethyl(meth)acrylate, in a weight ratio of adhesive component to non-adhesive component of about 1:25 to about 25:1.
c. Volatile solvent: isopropanol, in about 5% to about 40% by weight, and preferably about 5 to about 20% by weight.

### 2) Active Agents

a. Photodamage protective ingredient: zinc oxide (ZnO), in about 2% to about 25% by weight.
b. Photodamage reparative ingredient: enzogenol, in about 0% to about 15% by weight, but more preferably from about 0.1% to about 5% by weight.
c. Additional Topical Active: dihydroxyacetone in about 2% to about 5% concentration by weight.
d. Preservatives: none.

### 3) Aesthetic Modifying Agents

a. Pigments: standard pigments added to achieve a range of flesh-colored tones, in about 1% to about 15% by weight, but more preferably about 1% to about 8% by weight.
b. Rheological Agents: none.

The total suspended particles being present in the dispersion comprise about 1% to about 70% by weight.

Other embodiments may include other/additional contact-active ingredients, such as vitamins, medications, and the like.

Additional embodiments include a variety of forms comprising aerosol spray; or cream, gel or other film bases, which may be applied topically in a variety of different ways such as wiping or rubbing, to achieve the same goals.

### Sample Embodiment 2

### 1) Base

a. Adhesive component: polymer of about 60% to about 80% isooctyl acrylate, about 1% to about 10% acrylamide, and about 5% to about 30% vinyl acetate.
b. Non-adhesive component: polymethyl(meth)acrylate, in a weight ratio of adhesive component to non-adhesive component of about 1:25 to about 25:1.
c. Volatile solvent: isopropanol, in about 5% to about 40% by weight, and preferably about 5% to about 20% by weight.

### 2) Active Agents

a. Photodamage protective ingredient: titanium dioxide (TiO₂) nanoparticles, in about 1% to about 50% by weight.
b. Photodamage reparative ingredient: manuka oil, in about 0.1% to about 50% by weight, but more preferable from about 0.1% to about 10% by weight.
c. Additional Topical Active: dihydroxyacetone in about 2% to about 5% concentration by weight.
d. Preservatives: benzyl alcohol about 1% to about 8% by weight, but more preferably about 1% to about 3% by weight.

### 3) Aesthetic Modifying Agents

a. Pigments: standard pigments added to achieve a range of bright colors, in about 1% to about 15% by weight, but more preferably about 1% to about 8% by weight.
b. Rheological Agents: sodium hyaluronate, in about 1% to about 10% by weight, but more preferably about 1% to about 5% by weight.

The total suspended particles being present in the dispersion comprise about 1% to about 70% by weight.

### Sample Embodiment 3

### 1) Base

a. Adhesive component: polymer of about 60% to about 80% isooctyl acrylate, about 1% to about 10% acrylamide, and about 5% to about 30% vinyl acetate.
b. Non-adhesive component: polymethyl(meth)acrylate, in a weight ratio of adhesive component to non-adhesive component of about 1:25 to about 25:1.
c. Volatile solvent: Isopropanol, in about 5% to about 40% by weight, and preferably about 5% to about 20% by weight.

### 2) Active Agents

a. Photodamage protective ingredient 1: zinc oxide (ZnO), in about 0.5% to about 25% by weight.
b. Photodamage protective ingredient 2: titanium dioxide (TiO₂) nanoparticles, in about 0.5% to about 50% by weight.
c. Photodamage reparative ingredient: manuka oil, in about 0.1% to about 50% by weight, but more preferable from about 0.1% to about 10% by weight.
d. Additional Topical Active: dihydroxyacetone in about 2% to about 5% concentration by weight.
e. Preservatives: benzyl alcohol about 1% to about 8% by weight, but more preferably about 1% to about 3% by weight.

### 3) Aesthetic Modifying Agents

c. Pigments: standard pigments added to achieve a range of flesh colors and bright colors, in about 1% to about 15% by weight, but more preferably about 1% to about 8% by weight.
d. Rheological Agents: sodium hyaluronate, in about 1% to about 10% by weight, but more preferably about 1% to about 5% by weight.

The total suspended particles being present in the dispersion comprise about 1% to about 70% by weight.

### Sample Embodiment 4

To prepare 150 ml of total formulation we mixed 44.8 g of adhesive component (polymer of 75% isooctyl acrylate, 15% acrylamide, 10% vinyl acetate), with 29.2 g of the non-adhesive component (polymethyl(meth)acrylate), and 4 g of the volatile solvent isopropanol, and stirred until a smooth slurry was formed. After heating the mixture to 30 degrees Celsius, 15 g of micronized zinc oxide (ZnO) was added. Once evenly mixed (using commercial agitator×10 minutes), 2 g of the photodamage reparative agent enzogenol, 3 g of the topical active dihydroxyacetone (DHA), and 2 g of flesh-colored pigment were added, and the entire formula was again agitated to evenly disperse agents throughout (using paddle blade agitation×10 minutes). While cooling to room temperature, 50 ml water was then mixed into this final formulation using propeller agitation until homogenous.

This formula was then applied to 5 test subjects (right thigh) who each spent an average of 15 hours in the sun over a period of 4 days. The subjects were allowed to shower as usual using regular soap and water, and were involved in outdoor activities involving both land and ocean, without any restrictions. No other sunblocks or clothing was applied to the test sites during sun exposure for the duration of this pilot study. Treated R. sides were compared with untreated L. sides and showed no evidence of erythema (redness) or tanning occurring at the treatment sites. Control sites (left thigh) in contrast, demonstrated both tanning and erythema.

### Sample Embodiment 5

First, a commercially available micronized ZnO powder was applied to the surface of the test subject's skin of the right thigh in a 5×5 cm patch. Next, this area was marked with indelible pen (Sharpie™) to outline the treated area. Next, the same region was coated with a film of a commercially available base mixture of acrylates (including hexamethyldisiloxane, acrylate terpolymer, polyphenylmethylsiloxane copolymer) to evenly cover the entire area. The left thigh was not treated with sunblock or liquid bandage but a similar area was blocked off. The 5 test subjects spent an average of 15 hours in the sun each over a period of 4 days. The subjects were allowed to shower as usual using regular soap and water, and were involved in outdoor activities involving both land and ocean, without any restrictions. No other sunblocks or clothing was applied to the test sites during sun exposure for the duration of the pilot study. Treated and untreated sides were compared and showed no evidence of erythema (redness) or tanning occurring at the treatment sites. Control sites (left thigh) in contrast, demonstrated both tanning and erythema.

### Sample Embodiment 6

First, a commercially available 300 μm TiO₂ powder was applied to the surface of the test subject's skin of the right thigh in a 5×5 cm patch. This area was marked with indelible pen (Sharpie™) to outline the treated area. Next, the same region was coated with a film of a commercially available base mixture of acrylates, as used in sample embodiment 5, to evenly cover the entire area. The left thigh was not treated with sunblock or liquid bandage but a similar area was blocked off. The 5 test subjects spent an average of 15 hours each in the sun over a period of 4 days. The subjects were allowed to shower as usual using regular soap and water, and were involved in outdoor activities involving both land and ocean, without any restrictions. No other sunblocks or clothing was applied to the test sites during sun exposure for the duration of the pilot study. Treated and untreated sides were compared and showed no evidence of erythema (redness) or tanning occurring at the treatment sites. Control sites (left thigh) in contrast, demonstrated both tanning and erythema.

### Sample Embodiment 7

A commercially available base mixture of acrylates, as used in sample embodiment 5, was used as a representative test base (85% by weight), to which sodium benzotriazolyl butylphenol sulfonate (and) buteth-3 (and) tributyl citrate (4% by weight), micronized ZnO (0.5% by weight), microfine TiO₂ (0.5% by weight), tetrabutyl ethylidinebisphenol (4% by weight), and tris(tetramethylhydroxypiperidinol) citrate (4% by weight) were sequentially added, during paddle agitation to achieve a homogenous suspension.

The total suspended particles being present in the suspension comprise about 1% to about 70% by weight.

After shaking for 30 seconds the suspension sprayed evenly onto thigh skin and remained in place for >3 days in all of 4 subjects.

### Sample Embodiment 8

A commercially available base mixture of acrylates, as used in sample embodiment 5, was used as a representative test base (86% by weight), to which sodium benzotriazolyl butylphenol sulfonate (and) buteth-3 (and) tributyl citrate (4.5% by weight), micronized ZnO (0.5% by weight), microfine TiO₂ (0.5% by weight), octadecyl di-t-butyl-4-hydroxyhydrocinnamate (4.5% by weight), and tris (tetramethylhydroxypiperidinol) citrate (4% by weight) were sequentially added, during paddle agitation to achieve a homogenous suspension.

The total suspended particles being present in the suspension comprise about 1% to about 70% by weight.

After shaking for 30 seconds the suspension sprayed evenly onto thigh skin and remained in place for >3 days in all of 4 subjects.

### Sample Embodiment 9

A commercially available base mixture of acrylates, as used in sample embodiment 5, was used as a representative test base (88.5% by weight), to which sodium benzotriazolyl butylphenol sulfonate (4.4% by weight), micronized ZnO (0.4% by weight), microfine TiO₂ (0.4% by weight), tetrabutyl ethylidinebisphenol (2.3% by weight), and tris(tetramethylhydroxypiperidinol) citrate (4% by weight) were sequentially added, during paddle agitation to achieve a homogenous suspension.

The total suspended particles being present in the suspension comprise about 1% to about 70% by weight.

After shaking for 30 seconds the suspension sprayed evenly onto thigh skin and remained in place for >4 days in all of 4 subjects.

### Sample Embodiment 10

A commercially available base mixture of acrylates, as used in sample embodiment 5, was used as a representative test base (95% by weight), to which sodium benzotriazolyl butylphenol sulfonate (3% by weight), micronized ZnO (1% by weight), and microfine TiO₂ (1% by weight) were sequentially added, during paddle agitation to achieve a homogenous suspension.

The total suspended particles being present in the suspension comprise about 1% to about 70% by weight.

After shaking for 30 seconds the suspension sprayed evenly onto thigh skin and remained in place for >5 days in all of 4 subjects.

### Sample Embodiment 11

A commercially available base mixture of acrylates, as used in sample embodiment 5, was used as a representative test base (87% by weight), to which sodium benzotriazolyl butylphenol sulfonate (5% by weight), micronized ZnO (0.75% by weight), microfine TiO₂ (0.75% by weight), octadecyl di-t-butyl-4-hydroxyhydrocinnamate (1.5% by weight), and tris(tetramethylhydroxypiperidinol) citrate (5% by weight) were sequentially added, during paddle agitation to achieve a homogenous suspension.

The total suspended particles being present in the suspension comprise about 1% to about 70% by weight.

After shaking for 30 seconds the suspension sprayed evenly onto thigh skin and remained in place for >5 days in all of 4 subjects.

### Sample Embodiment 12

A commercially available base mixture of acrylates, as used in sample embodiment 5, was used as a representative test base (93% by weight), to which sodium benzotriazolyl butylphenol sulfonate (2.4% by weight), micronized ZnO (3.3% by weight), and octadecyl di-t-butyl-4-hydroxyhydrocinnamate (1.3% by weight) were sequentially added, during paddle agitation to achieve a homogenous suspension.

The total suspended particles being present in the suspension comprise about 1% to about 70% by weight.

After shaking for 30 seconds the suspension sprayed evenly onto thigh skin and remained in place for >4 days in all of 4 subjects.

### Sample Embodiment 13

A commercially available base mixture of acrylates, as used in sample embodiment 5, was used as a representative test base (93% by weight), to which sodium benzotriazolyl butylphenol sulfonate (2.4% by weight), microfine TiO₂ (3.3% by weight), and octadecyl di-t-butyl-4-hydroxyhydrocinnamate (1.3% by weight) were sequentially added, during paddle agitation to achieve a homogenous suspension.

The total suspended particles being present in the suspension comprise about 1% to about 70% by weight.

After shaking for 30 seconds the suspension sprayed evenly onto thigh skin and remained in place for >4 days in all of 4 subjects.

### Sample Embodiment 14

A commercially available base mixture of acrylates, as used in sample embodiment 5, was used as a representative test base (89.5% by weight), to which methylene bis-benzotriazolyl tetramethylbutylphenol (4% by weight), octadecyl di-t-butyl-4-hydroxyhydrocinnamate (1.5% by weight), and tris (tetramethylhydroxypiperidinol) citrate (5% by weight) were sequentially added, during paddle agitation to achieve a homogenous suspension.

The total suspended particles being present in the suspension comprise about 1% to about 70% by weight.

After shaking for 30 seconds the suspension sprayed evenly onto thigh skin and remained in place for >5 days in all of 4 subjects.

### Sample Embodiment 15

A commercially available base mixture of acrylates, as used in sample embodiment 5, was used as a representative test base (91% by weight), to which butyl methoxydibenzoylmethane (2.5% by weight), octadecyl di-t-butyl-4-hydroxyhydrocinnamate (1.5% by weight), and tris(tetramethylhydroxypiperidinol) citrate (5% by weight) were sequentially added, during paddle agitation to achieve a homogenous suspension.

The total suspended particles being present in the suspension comprise about 1% to about 70% by weight.

After shaking for 30 seconds the suspension sprayed evenly onto thigh skin and remained in place for >5 days in all of 4 subjects.

### Sample Embodiment 16

To prepare 150 ml of total formulation we mixed 39.6 g of adhesive component (polymer of 68% isooctyl acrylate, 18% acrylamide, 14% vinyl acetate) into 50 ml water using propeller agitation until homogenous. Next, we added 28.9 g of non-adhesive component (polymethyl(meth)acrylate), and 4 g of the volatile solvent isopropanol, and mixed this solution using a commercial paddle blade agitator×10 minutes while heating the mixture to 30 deg Celsius. Next, we added 15 g of 200 μm ground TiO₂ (our preferred photoprotective agent for this formulation), 2 g of the photodamage reparative agent manuka oil, 3 g of the topical active dihydroxyacetone (DHA), and 2 g of flesh-colored pigment while continuing agitation at 30 deg Celsius until suspended particles were evenly dispersed throughout. Before cooling, 3.8 g of the preservative benzyl alcohol, and 1.7 g of the rheological agent sodium hyaluronate were added and again mixed by agitation to even dispersion. Thereafter the formulation was cooled to room temperature. This formula was then applied to 5 test subjects (right thigh) who spent an average of 15 hours in the sun each over a period of 4 days. The subjects were allowed to shower, using usual soap and water regimens, and were involved in outdoor activities involving both land and ocean, without any restrictions. No other sunblocks or clothing was applied to the test sites during sun exposure for the duration of the pilot study. Treated R. sides were compared with untreated L. sides and showed minimal evidence of erythema (redness) or tanning at the treatment sites, while the control sites (left thigh) in contrast demonstrated significant tanning and erythema.

1-47. (canceled)

48. A broad-spectrum photoprotective composition that blocks or attenuates ultraviolet radiation within the range of 290 to 400 nm comprising:
(a) a base comprising
(i) an adhesive component comprising a first polymer that is a terpolymer of a C₄-C₁₀ alkyl(meth)acrylate and two comonomers selected from the group consisting of acrylates and (meth)acrylates having an esterified or nonesterified carboxyl group, unsubstituted or N-substituted acrylamides and (meth)acrylamides, and vinyl acetate;
(ii) a film-forming non-adhesive component comprising a second polymer; and
(iii) at least one volatile solvent; and
(b) a dispersion of one or more metal oxide particles selected from
(i) TiO₂ particles, at a concentration from 0.5 to 50% by weight of the broad-spectrum photoprotective composition;
(ii) ZnO particles, at a concentration from 0.5 to 25% by weight of the broad-spectrum photoprotective composition; and
(iii) mixtures thereof.

49. The broad-spectrum photoprotective composition of claim 48 wherein upon or after application of the photoprotective composition to a user's skin, the adhesive component and the film-forming non-adhesive component undergo phase separation and the adhesive component adheres to the outer layers of the stratum corneum of the user's skin.

50. The broad-spectrum photoprotective composition of claim 49 wherein the adhesive component adheres to the outer layers of the stratum corneum for 8 to 96 hours as determined by histological testing using the hematoxylin and eosin stain method.

51. The broad-spectrum photoprotective composition of claim 49 wherein the adhesive component adheres to the outer layers of the stratum corneum for 7 days as determined by histological testing using the hematoxylin and eosin stain method.

52. The broad-spectrum photoprotective composition of claim 49 wherein the adhesive component adheres to the outer layers of the stratum corneum for 14 days as determined by histological testing using the hematoxylin and eosin stain method.

53. The broad-spectrum photoprotective composition of claim 48 wherein the weight ratio between the adhesive component to the film-forming non-adhesive component is 8:1 to 1:8.

54. The broad-spectrum photoprotective composition of claim 48 wherein the first polymer is a terpolymer of isooctyl (meth)acrylate and two monomers selected from the group consisting of 2-ethoxyethyl methacrylate, vinyl acetate, and acrylamide.

55. The broad-spectrum photoprotective composition of claim 48 wherein the second polymer is selected from the group consisting of: a polyphenylmethyl siloxane, silicone-polyurea block copolymers, silicone-polyurethane block copolymers, polyl(methyl methacrylate), fluorinated polymers, fluorosilicone polymers, ethyl cellulose, nitrocellulose, and a combination of these.

56. The broad-spectrum photoprotective composition of claim 48 wherein the metal oxide particles are micronized to diameters of 1,000 μm or less.

57. The broad-spectrum photoprotective composition of claim 56 wherein the metal oxide particles are ZnO.

58. The broad-spectrum photoprotective composition of claim 56 further comprising one or more organic chemical sunscreens.

59. The broad-spectrum photoprotective composition of claim 58 wherein the organic chemical sunscreen is selected from the group of octyl methoxycinnamate and avobenzone.

60. The broad-spectrum photoprotective composition of claim 59 wherein the organic chemical sunscreen is avobenzone and the composition further comprises an excited state quencher.

61. The broad-spectrum photoprotective composition of claim 48 further comprising at least one of a photodamage reparative ingredient selected from the group consisting of vitamins, cofactors, minerals, antioxidants and quenchers.

62. A broad-spectrum photoprotective composition comprising:
(a) a base comprising
(i) a cyanoacrylate monomer;
(ii) a film-forming non-adhesive component comprising a second polymer; and
(iii) at least one volatile solvent;
(b) a dispersion of one or more metal oxide particles comprising
(i) TiO₂ particles, at a concentration from 0.5 to 50% by weight of the broad-spectrum photoprotective composition;
(ii) ZnO particles, at a concentration from 0.5 to 25% by weight of the broad-spectrum photoprotective composition; and
(iii) mixtures thereof.

63. The broad-spectrum photoprotective composition of claim 62 wherein the adhesive component adheres to the outer layers of the stratum corneum for 8 to 96 hours as determined by histological testing using the hematoxylin and eosin stain method.

64. The broad-spectrum photoprotective composition of claim 62 wherein the adhesive component adheres to the outer layers of the stratum corneum for 7 days as determined by histological testing using the hematoxylin and eosin stain method.

65. The broad-spectrum photoprotective composition of claim 62 wherein the adhesive component adheres to the outer layers of the stratum corneum for 14 days as determined by histological testing using the hematoxylin and eosin stain method.

66. The broad-spectrum photoprotective composition of claim 62 wherein the cyanoacrylate monomer is 2-octyl cyanoacrylate.

67. The broad-spectrum photoprotective composition of claim 62 wherein the second polymer is selected from the group consisting of: a polyphelylmethyl siloxane, silicone-polyurea block copolymers, silicone-polyurethane block copolymers, polyl(methyl methacrylate), fluorinated polymers, fluorosilicone polymers, ethyl cellulose, nitrocellulose, and a combination of these.

68. The broad-spectrum photoprotective composition of claim 62 wherein the metal oxide particles are micronized to diameters of 1,000 μm or less.

69. The broad-spectrum photoprotective composition of claim 68 wherein the metal oxide particles are ZnO.

70. The broad-spectrum photoprotective composition of claim 68 further comprising one or more organic chemical sunscreens.

71. The broad-spectrum photoprotective composition of claim 70 wherein the organic chemical sunscreen is selected from the group of octyl methoxycinnamate and avobenzone.

72. The broad-spectrum photoprotective composition of claim 71 wherein the organic chemical sunscreen is avobenzone and the composition further comprises an excited state quencher.

73. The broad-spectrum photoprotective composition of claim 62 further comprising at least one of a photodamage reparative ingredient selected from the group consisting of vitamins, cofactors, minerals, antioxidants and quenchers.

74. A method of providing protection to human skin from photodamage caused by exposure to ultraviolet radiation within the range of 290 nm to 400 nm comprising the step of applying to the human skin the composition of claim 48.

75. The method of providing protection to human skin from photodamage of claim 74 wherein the protection is provided for a period of 8 to 96 hours.

76. A method of providing protection to human skin from photodamage caused by exposure to ultraviolet radiation within the range of 290 nm to 400 nm comprising the step of applying to the human skin the composition of claim 62.

77. The method of providing protection to human skin from photodamage of claim 76 wherein the protection is provided for a period of 8 to 96 hours.
